(19) United States
(12) Patent Application Publication
(10) Pub. No.: US 2005/0182348 A1
(43) Pub. Date: Aug. 18, 2005

(54) Support system in bras, pants, girdles, apparel braces, etc. made up of an insert, harness and/or zipper

(76) Inventor: Joseph O. Smith, Wellington, FL (US)

Correspondence Address: DR. JOSEPH SMITH;DOCTOR'S CORNER SPORT SUPPORT, 1921 S. CLUB DRIVE, WELLINGTON, FL, 33414, US

(21) Appl. No.: 10/932,357

(22) Filed: Aug. 16, 2004

Related U.S. Application Data
(63) Continuation-in-part of application No. 09/798,209, filed on Mar. 5, 2001, now Pat. No. 6,776,769.
(63) Continuation-in-part of application No. 10/227,654, filed on Aug. 26, 2002, now abandoned.
Application No. 10/227,654 is a continuation-in-part of application No. 11/029,160.

(51) Int. Cl. A61F 13/06; A61F 13/00

(52) U.S. Cl. 602/61

(57) ABSTRACT

Our device is made of different materials that may stretch in four directions, aiding in support and comfort. The overlapping cylinder like shapes provide 3-D, 360° support to the anatomical area to which they are placed.

### BACKGROUND OF THE INVENTION

1. Field of the Invention

This invention relates to an article of manufacture. More specifically, this invention is directed to a device and method for supporting an articulating joint, joints, and/or body part (e.g. wrist, ankle, elbow, knee, low back, full body, upper back, foot, and back of the neck and head). In the preferred embodiments of this invention, the device comprises spherically, round and/or oval (like) overlapping cylinder like shapes placed in braces, clothing, and footwear, designed as a pillow, and placed in a seat or furniture for orthopedic support.

2. Description of the Prior Art

The human body may be injured by strains, contusions, bruises, degenerative joint diseases, arthritis, and arthroscopic operations. Such injuries may cause hematomas and inflamed areas that need to be treated. In such cases, it has been found advantageous to apply a compress of some type to the injured area to effect more rapid healing, or prevent any further inflammation or injury. Typically, support for the joints can be provided with an Ace bandage wrap, or an anatomically configured tubular device. The anatomically configured tubular device can have an opening in the device at the heel, to permit greater of lesser degree of movement of the ankle. Similarly, a wrist support may include a sleeve having one or more finger or thumb loops to prevent relative movement (creep) of the support on the wrist while in use.

A number of patents have been disclosed in the patent literature which discloses various devices for support or treatment of a weakened or injured articulating joint. The following patents are discussed in chronological order and, thus, no significance is to be attached to their order of discussion.

### OBJECTS OF THE INVENTION

It is the object of this invention to remedy the above as well as related deficiencies in the prior art.

More specifically, it is the principal object of the present invention to provide an anatomically configured device for pressure support of an articulating joint, joints or body fitted part.

It is another object of this invention to provide an anatomically configured device for pressure support of an articulating joint, joints or body fitted part, which may stretch inferior, superior, medial and lateral.

It is yet another object of this invention to provide an anatomically configured device of material within a brace, clothing, footwear, as a pillow, a seat for concentrated pressure 360° support of an articulating joint, joints or body fitted part.

### SUMMARY OF THE INVENTION

The above and related objects are achieved by providing a device made of overlapping cylinder like shapes giving an enhanced 360° support over the joint, joints and/or body part in question within the brace, clothing, footwear, seats, as a pillow, which may stretch in four directions for pressure support of an ankle, elbow, knee, wrist, low back, full body, upper back, and/or neck and head. The device of this invention can include an anatomically configured device, which is used in one or more areas of the brace, clothing, footwear, seat, used as a pillow, etc., providing a 360° support, so as to focus or concentrate pressure support at the inferior, superior, medial and lateral points.

In the preferred embodiments of this invention, the pressure concentrating means of the composite comprises a delimited area having a plurality of cylinder like shapes of materials that could have relatively greater stretch resistance and/or support, than other devices used for orthopedic support. For example, in the case of a device for support of a wrist, the pressure concentrating means can be located either on the device just forward of the wrist on either the palm or backside of the hand.

In another of the preferred embodiments of this invention, the composite device of this invention can include one or multiple delmited areas of a brace, clothing, footwear, seat, or as a pillow with a concentrating 360° support for support to the body. For example, in the case of a device for pressure support of the ankle, the pressure support means are typically positioned to both support the Achilles tendon and the plantar of the foot.

The device of this invention can also be provided in the form of overlapping cylinder like shapes providing a 360° support on a brace, clothing, footwear, seat, or as a pillow.

### BRIEF DESCRIPTION OF THE DRAWINGS

FIG. 1 is a perspective view of the full body support of this invention.

FIG. 2 is a perspective view of a Sports Bra device of this invention.

FIG. 3 is a perspective view of a pillow device of this invention.

FIG. 4 is a perspective view of footwear with the device of this invention.

FIG. 5 is a perspective view of a car seat with the support device of this invention.

### DETAILED DESCRIPTION OF THE INVENTION INCLUDING PREFERRED EMBODIMENTS

The Figures which accompany this application, and referenced herein, depict representative embodiments of this invention. In each instance, the composite comprises layers of material with overlapping cylinder like shapes providing 3-D, 360° support having concentrated pressure support means locations for pressure support in braces, clothing, footwear, pillows and/or seats. The layers of material can be anatomically placed at a particular location of the body, providing 3-D, 360° support within a brace, and may also stretch in four directions.

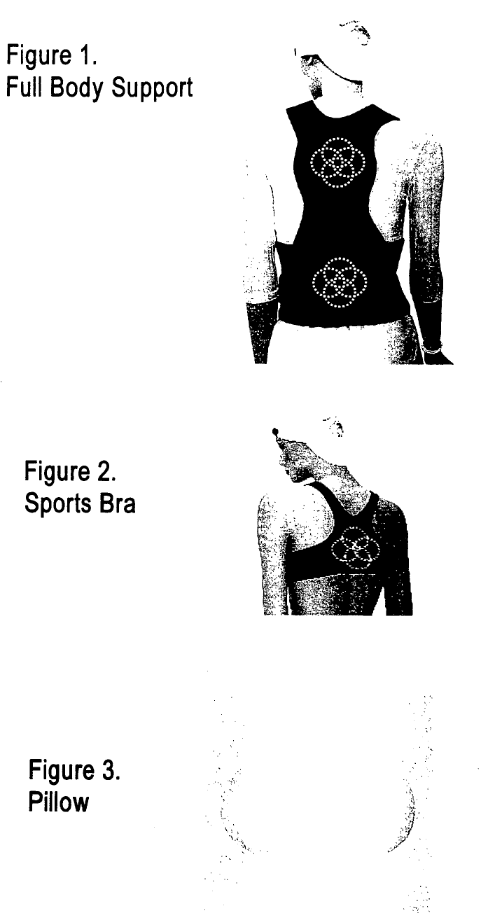

In FIG. 1, the devices of this invention are preconfigured in an anatomical position suitable to support the upper and low back.

Similarly, in FIG. 2, the device is preconfigured in an anatomical position suitable to support the upper back as it is integrated in a pullover sports bra. This device supports the upper dorsal area

FIG. 3 illustrates the adaptation of the concepts of this invention to a device as a cervical/lumbar pillow that is anatomically configured suitable for support of the neck posteriorly, laterally, as well as low back posteriorly.

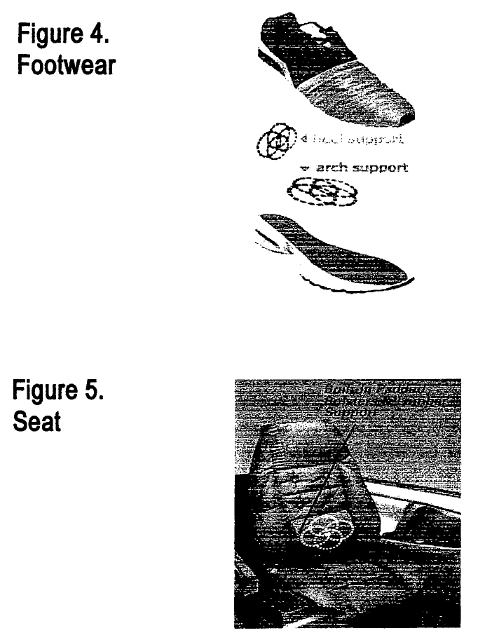

FIG. 4 illustrates the concept of this invention and the placement of these two designs for support and protection of the arch of the foot inferiorly and the ankle of the foot posteriorly.

FIG. 5 illustrates the concept of this device being integrated in a car seat to provide the lumbar support and comfort.

The pressure concentrating means depicted in each of the foregoing devices comprises a plurality of overlapping cylinder like shapes of material that may have greater stretch resistance than the material of the object they are placed in.

In each case, the combined effect of the pressure concentrating means and the material of the devices providing 3-D, 360° support at specific areas of the devices of this invention is to lend additional support or focused pressure to the brace, clothing, footwear, pillow, and/or seat at one or more locations.

1. The device placed in the full body support (FIG. 1) provides pressure support in the upper dorsal and low back/lumbar areas.
The device comprises a composite of overlapping cylinder like shapes within the brace and at least one pressure concentrating means located at a predesignated areas on the device, the pressure concentrating means comprising at least one component which when combined with the brace, lends additional and/or enhanced support or focused pressure at one or more locations on the device where the pressure concentrating means are located.

2. The device placed in clothing (FIG. 2) offers an enhanced support and comfort to individuals in the upper back areas.

3. The device when used as a pillow support (FIG. 3) provides an adaptable, ergonomically designed pillow for support and comfort for the head and neck while sleeping, and supports the low back when used on a chair or seat at home, in the car, or at work.

4. The design when anatomically configured to the footwear (FIG. 4) provides increased focal support in the arch of the foot inferiorly and ankle of the foot posteriorly.

5. The design when anatomically configured to the seat (FIG. 5) or chair provides increased focal support to the lumbar spine aiding to an enhanced comfort and stability.
